(19) United States
(12) Patent Application Publication
(10) Pub. No.: US 2025/0275733 A1
(43) Pub. Date: Sep. 4, 2025

(54) IMAGING SYSTEM WITH COMPOSITE GEARING AND METHOD THEREOF

(71) Applicant: Hologic, Inc., Marlborough, MA (US)

(72) Inventor: Ben W. Fagen, JR., Enfield, CT (US)

(73) Assignee: Hologic, Inc., Marlborough, MA (US)

(21) Appl. No.: 19/055,186

(22) Filed: Feb. 17, 2025

Related U.S. Application Data
(60) Provisional application No. 63/559,889, filed on Mar. 1, 2024.

(51) Int. Cl. A61B 6/00; A61B 6/02; A61B 6/04; A61B 6/50

(52) U.S. Cl. CPC A61B 6/4476; A61B 6/025; A61B 6/0414; A61B 6/0421; A61B 6/4435; A61B 6/502

(57) ABSTRACT

An x-ray imaging system is configured for mammography and tomosynthesis imaging modes. A rotational drive system is configured to support and drive rotational movement of an x-ray tube arm and a compression arm. A gearing system includes a driving gear and a driven gear. The driven gear is a composite gear with a tooth ring formed from a polymer material. The driving gear and the driven gear are in an over-meshed configuration such that a loading force is induced within the teeth of the driven gear engaged with the driving gear. The loading force reduces or prevents lash movement of the x-ray tube arm during rotation into and out of a 0° tube arm angle position.

### CROSS-REFERENCE TO RELATED APPLICATION(S)

This application claims priority to and the benefit of U.S. Provisional Patent Application No. 63/559,889, filed Mar. 1, 2024, which is incorporated herein by reference in its entirety.

### BACKGROUND

Breast cancer and other breast lesions continue to be a significant threat to women's health. X-ray mammography and tomosynthesis are the most widely used tools for carly detection, screening, and diagnosis. At least some known imaging systems that perform mammography and tomosynthesis imaging procedures have an upright gantry with an x-ray tube arm that at least partially rotates and a compression arm that at least partially rotates. A gear box with a gear system is typically used to facilitate the rotational movement of the x-ray tube arm and the compression arm. The gear system includes metal gears meshed with each other and with clearance between the gears so that lubrication can flow therebetween. As the x-ray tube arm and/or the compression arm rotates, the clearance between the gears can induce angular discontinuity jumps of the x-ray tube arm and/or compression arm because of positional variations in the supported weight. Accordingly, improvements to the rotational drive system of the x-ray tube arm and/or compression arm are desired.

### SUMMARY

Examples of the disclosure are directed to composite gear systems for rotational drive assemblies in mammography and tomosynthesis imaging systems.

In an aspect, the technology relates to an x-ray imaging system including: a gantry including a rotational drive assembly; an x-ray tube arm including an x-ray source coupled to the gantry at the rotational drive assembly, the x-ray tube arm configured to selectively rotate relative to the gantry around a rotational axis via the rotational drive assembly; and an arm including an immobilization system coupled to the gantry and independently rotatable relative to the gantry around the rotational axis via the rotational axis, wherein the immobilization system includes a paddle, a support platform, and an x-ray receptor disposed below the support platform, wherein the rotational drive assembly includes: a tube shaft rotatable around the rotational axis and supporting the x-ray tube arm; an actuator configured to drive rotation of the tube shaft; and a gearset including a driving gear coupled to the actuator and having at least one first tooth and a driven gear coupled to the tube shaft and having at least one second tooth, wherein the at least one second tooth of the driven gear is formed from a polymer material, and wherein the driven gear is over-meshed with the driving gear such that the at least one second tooth is in direct contact with the at least one first tooth with the at least one second tooth at least partially deflected causing a loading force within the at least one second tooth.

In an example, the deflection of the at least one second tooth includes bending and compression of the at least one second tooth. In another example, the loading force is between 0.1 kilopound per square inch (ksi) and 5 ksi. In still another example, the loading force is measured when the x-ray tube arm is at a 0° tube arm angle. In yet another example, the loading force occurs when the x-ray tube arm is between at least a ±5° and a ±30° tube arm angles. In an example, the driving gear is formed from a metal material, the driving gear is a wormshaft and the at least one first tooth is a helical tooth.

In another example, the driving gear includes a metal core, the polymer material cast on the metal core and forming a tooth ring. In still another example, the rotational drive assembly includes a second tube shaft supporting the arm, a second actuator configured to drive rotation of the second tube shaft, and a second gearset, the second gearset having a smaller diameter driven gear than the driven gear of the tube shaft of the x-ray tube arm. In yet another example, the driven gear is rotatable around the rotational axis and the driving gear is rotatable around a drive axis, and a distance between the drive axis and the rotational axis is such that the driving gear has an interference fit with the driven gear and a first pitch radius of the at least one first tooth at least partially overlaps with a second pitch radius of the at least one second tooth. In an example, the overlap of the first pitch radius of the at least one first tooth and the second pitch radius of the at least one second tooth compresses the at least one second tooth inducing the loading force therein. In another example, the overlap of the first pitch radius of the at least one first tooth and the second pitch radius of the at least one second tooth is between 1% and 5% of the distance between the drive axis and the rotational axis at a 0° tube arm angle in a fully-meshed configuration.

In still another example, the interference fit is measured when the x-ray tube arm is at a 0° tube arm angle. In yet another example, the interference fit occurs when the x-ray tube arm is between at least a ±5° and a ±30° tube arm angles.

In another aspect, the technology relates to a method of rotating an x-ray tube arm of an imaging system, the imaging system including a gantry rotationally supporting the x-ray tube arm and an arm having an immobilization system including a paddle, a support platform, and an x-ray receptor disposed below the support platform, the method including: providing a rotational drive assembly, wherein the rotational drive assembly includes a tube shaft rotatable around a tube arm axis and supporting the x-ray tube arm, an actuator, and a gearset including a driving gear coupled to the actuator and having at least one first tooth and a driven gear coupled to the tube shaft and having at least one second tooth, wherein the at least one second tooth of the driven gear is formed from a polymer material, and wherein the driven gear is over-meshed with the driving gear such that a loading force is induced within the at least one second tooth; positioning, via the rotational drive assembly, the x-ray tube arm that includes an x-ray source at a 0° tube arm angle relative to the gantry; rotating the x-ray tube arm about the tube arm axis and out of the 0° tube arm angle by actuating the driving gear thereby rotating the driven gear around the tube arm axis; and based on the loading force within the at least one second tooth, preventing lash of the x-ray tube arm via the driven gear when a weight of the x-ray tube arm moves out of the 0° tube arm angle.

In an example, the method further includes inducing the loading force based on a force-displacement approach, a pressure approach, or a sensing approach. In another example, rotating the x-ray tube arm occurs during a tomosynthesis imaging mode of the imaging system. In still another example, the loading force prevents lash of the x-ray tube arm at least between a ±5° and a ±30° tube arm angles.

In another aspect, the technology relates to an x-ray imaging system including: a gantry including a rotational drive assembly defining a rotational axis; an x-ray tube arm including an x-ray source coupled to the gantry at the rotational drive assembly, the x-ray tube arm configured to selectively rotate relative to the gantry via the rotational drive assembly; and an arm including an immobilization system coupled to the gantry at the rotational drive assembly and independently rotatable relative to the gantry, wherein the immobilization system includes a paddle, a support platform, and an x-ray receptor disposed below the support platform, wherein the rotational drive assembly includes: a tube shaft rotatable around the rotational axis and supporting the x-ray tube arm; an actuator configured to drive rotation of the tube shaft; and a gearset including a driving gear coupled to the actuator and having at least one first tooth and a driven gear coupled to the tube shaft, rotatable around the rotational axis, and having at least one second tooth, wherein the at least one second tooth of the driven gear is formed from a polymer material, wherein the driving gear is rotatable around a drive axis, and wherein a distance between the drive axis and the rotational axis is such that the driving gear has an interference fit with the driven gear and a first pitch radius of the at least one first tooth at least partially overlaps with a second pitch radius of the at least one second tooth.

In an example, the overlap of the first pitch radius of the at least one first tooth and the second pitch radius of the at least one first tooth compresses the at least one second tooth inducing a loading force therein. In another example, the overlap of the first pitch radius of the at least one first tooth and the second pitch radius of the at least one second tooth is between 1% and 5% of the distance between the drive axis and the rotational axis at a 0° tube arm angle in a fully-meshed configuration. In still another example, the interference fit is measured when the x-ray tube arm is at a 0° tube arm angle. In yet another example, the interference fit occurs when the x-ray tube arm is between at least a ±5° and a ±30° tube arm angles. In an example, the driving gear is formed from a metal material, the driving gear is a wormshaft and the at least one first tooth is a helical tooth.

In another example, the driving gear includes a metal core, the polymer material cast on the metal core and forming a tooth ring. In still another example, the rotational drive assembly includes a second tube shaft supporting the arm, a second actuator configured to drive rotation of the second tube shaft, and a second gearset, the second gearset having a smaller diameter driven gear than the driven gear of the tube shaft of the x-ray tube arm.

### BRIEF DESCRIPTION OF DRAWINGS

The following drawings are illustrative of particular examples of the present disclosure and therefore do not limit the scope of the present disclosure. The drawings are not to scale and are intended for use in conjunction with the explanations in the following detailed description.

Examples of the present disclosure will hereinafter be described in conjunction with the appended drawings, wherein like numerals denote like elements.

FIG. 1 is a schematic view of an exemplary imaging system.

FIG. 2 is a perspective view of the imaging system of FIG. 1.

FIG. 3 is a schematic view of a compression arm and a tube arm of the imaging system shown in FIGS. 1 and 2.

FIG. 4 is a front perspective view of a rotational drive assembly for rotation of the compression arm and the tube arm shown in FIG. 3.

FIG. 5 is a rear perspective view of the rotational drive assembly of FIG. 4.

FIG. 6 is a cross-sectional, perspective view of a tube shaft assembly of the rotational drive assembly shown in FIGS. 4-5.

FIG. 7 is a perspective view of gearsets of the drive assembly shown in FIGS. 4-5.

FIG. 8 is a cross-sectional schematic view of a tube arm gearset of the gearsets shown in FIG. 7.

FIG. 8A is an enlarged tooth detail of the tube arm gearset shown in FIG. 8.

FIG. 9 is a flowchart illustrating an exemplary method of rotating an x-ray tube arm of an imaging system.

### DETAILED DESCRIPTION

Various examples will be described in detail with reference to the drawings, wherein like reference numerals represent like parts and assemblies throughout the several views. Reference to various examples does not limit the scope of the claims attached hereto. Additionally, any examples set forth in this specification are not intended to be limiting and merely set forth some of the many possible examples for the appended claims.

References in the specification to “one embodiment,” “an embodiment,” “an illustrative embodiment,” “an example,” “an aspect,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other examples whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C). Moreover, one having skill in the art will understand the degree to which terms such as “about,” “approximately,” or “substantially” convey in light of the measurement techniques utilized herein. To the extent such terms may not be clearly defined or understood by one having skill in the art, the term “about” shall mean plus or minus ten percent.

Throughout this description, references to orientation (c.g., front(ward), rear(ward), top, bottom, back, right, left, upper, lower, etc.) of the components of the imaging system relate to their position when installed on and in use with the imaging system and are used for case of description and illustration only. No restriction is intended by use of the terms regardless of how the components of the imaging system are situated on their own. As used herein, the terms “axial” and “longitudinal” refer to directions and orientations, which extend substantially parallel to a centerline of the component or system. Moreover, the terms “radial” and “radially” refer to directions and orientations, which extend substantially perpendicular to the centerline of the component or system. In addition, as used herein, the term “circumferential” and “circumferentially” refer to directions and orientations, which extend arcuately about the centerline of the component or system.

X-ray imaging systems include an x-ray tube arm rotating around an axis for various imaging modes or procedures. In a more specific example, digital breast tomosynthesis (c.g., DBT) relies on the x-ray tube arm moving in a limited arc angle over a stationary target while sequential projection images are captured and then reconstructed to provide information about cancers obscured by normal tissue located above and below a lesion. The limited arm movement may include a sweep in the range of 11° to 60°. A stable movement of the x-ray tube arm without discontinuities in the motion profile are desired to facilitate clear and accurate projection images. Known drive systems for x-ray tube arms have an all-metal worm gearing system. The metal gears are set to have clearance between them for lubrication and assembly tolerances. This clearance induces backlash or lash movement of the x-ray tube arm during rotational movement. For example, when the x-ray tube arm moves out of a 0° tube arm angle position, the weight of the x-ray tube arm causes the x-ray tube arm to undesirably move though the clearance within the gears (e.g., lash travel) and may cause angular motion discontinuity (c.g., image artifacts) in the projection images. Wear also occurs within the metal gears, thereby further increasing lash distances and requiring periodic service for re-lubrication and re-adjustment.

As used herein, 0° tube arm angle position is in relation to the tube arm being at a 12 o'clock position on the gantry because the lash travel is relative to the position of the tube arm on the gantry. It is appreciated that when describing imaging positions of the tube arm a 0° angle may also be used that relates to the object being imaged or the detector and the position of the tube arm such that the x-ray beam intersects object being image or the detector at a substantially 90° angle. When describing imaging positions, the 0° angle and the corresponding position of the tube arm may vary depending on the type of view. For example, in an MLO view, the tube arm position starts at a 30°-60° angle relative to the gantry.

Worm gearing provides a number of benefits for x-ray imaging systems because it is robust, quiet, and at high numeric ratios, self-locking or resistant to back driving. Additionally, x-ray tube arm motion is typically low speed, high-torque, and reversible. In the examples described herein, an all metal worm gearing system is replaced with a composite gearing system so as to maintain the benefits of the worm gearing system described herein. Additionally, the composite gearing system facilitates a reduction in noise during operation due to vibration damping, and additional efficiencies (e.g., torque transmission) due to lower friction and self-lubricating properties. A driving gear, the wormshaft, is a metal (e.g., steel) gear with a helical tooth form. The driven gear, the wormgear, is a composite gear with a polymer wormgear tooth ring secured to a steel core by a casting process. This composite gear system can operate without maintenance lubrication and achieves reduced tooth wear due to the reduced friction and the elastic properties of the polymer. In operation, a one-time break-in lubrication may be used for the composite gears during the initial operational period.

When merely replacing the all-metal gears with the composite gears and maintaining the typical tooth clearance spacing configuration of the gears, typically 0.002-0.004 inches, angular motion discontinuity can still occur in the imaging system. When the x-ray tube arm moves out of the 0° tube arm angle, the weight of the x-ray tube arm induces lash movement through the clearance spacing and image artifacts may occur. However, because of the material properties of the polymer teeth of the wormgear, additional gear tooth spacing configurations are enabled that can address the angular motion discontinuities induced by the inherent lash generated by the typical clearance spacing.

In operation, the loading on the gears is not uniform, and varies from essentially zero at the 0° tube arm angle position to a maximum load occurring at a ±90° tube arm angle position. While performing static overload testing with the composite wormgear set at the typical clearance spacing, it was observed that the angular deflections of the x-ray tube arm were completely reversible (e.g., elastic). These results are due to: (1) Wormgear tooth deflection from bending; (2) Wormgear tooth compression due to normal force between the metal wormshaft face and the contact patch on the plastic wormgear tooth; and (3) A shear gradient in the gear tooth ring acting as a windup mechanism between the gear tooth ring pitch circle and the metal core. Accordingly, by utilizing the elastic properties of the composite gear, the composite gear can be positioned relative to the wormshaft such that a loading force is induced within the wormgear teeth, and angular motion discontinuities of the tube arm are reduced or prevented when rotating out of the 0° tube arm angle position.

In examples, the loading force within the wormgear teeth can be predetermined for reducing or preventing lash movement of the x-ray tube arm at certain angular ranges, and for example, ±3°, ±7.5°, ±15°, ±30°, or more. This configuration reduces or prevents wear or additional stresses on the gears while maintaining reasonable lash movement of the x-ray tube arm at large angular ranges and that are more easily accounted for by load position encoders within the imaging system.

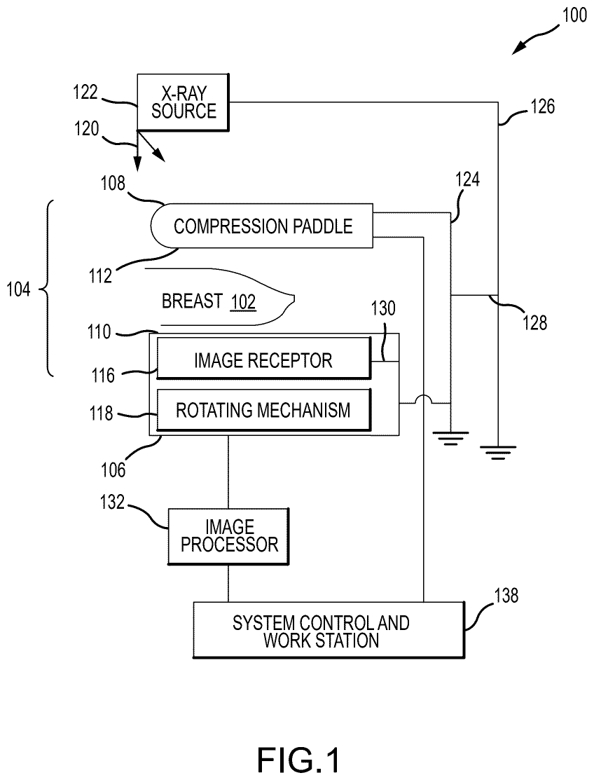

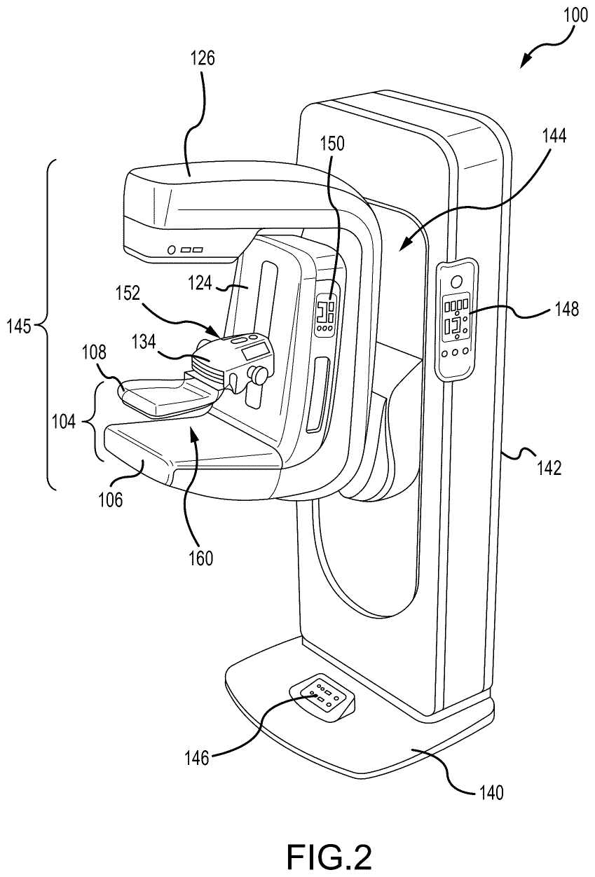

FIG. 1 is a schematic view of an exemplary imaging system 100. FIG. 2 is a perspective view of the imaging system 100. Referring concurrently to FIGS. 1 and 2, not every element described below is depicted in both figures. The imaging system 100 immobilizes a patient's breast 102 for x-ray imaging (either or both of mammography, tomosynthesis, or other imaging modalities) via a breast immobilizer unit 104 that includes a static breast support platform 106 and a moveable paddle 108. Different paddles, cach having different purposes, are known in the art. The breast support platform 106 and the paddle 108 cach have a compression surface 110 and 112, respectively, that move towards each other to compress, immobilize, stabilize, or otherwise hold and secure the breast 102 during imaging procedures. In known systems, the compression surface 110, 112 is exposed so as to directly contact the breast 102. Either or both of these compression surfaces 110, 112 may be rigid plastic, a flexible plastic, a resilient foam, a mesh or screen, and so on. The support platform 106 also houses an image receptor 116 and, optionally, a detector rotating mechanism 118, and optionally an anti-scatter grid (not depicted, but disposed above the image receptor 116). The immobilizer unit 104 is in a path of an imaging beam 120 emanating from an x-ray source 122, such that the beam 120 impinges on the image receptor 116.

The immobilizer unit 104 is supported on a first support arm, also referred to as a compression arm 124. The paddle 108 is coupled to a paddle arm 134 that is configured to be raised and lowered along the compression arm 124 forming an immobilization area 160 for the breast 102. The x-ray source 122 is supported on a second support arm, also referred to as a tube arm 126. For mammography, the compression arm and tube arm 124, 126 can rotate as a unit about an axis 128 between different imaging orientations such as CC and MLO, so that the imaging system 100 can take a mammogram projection image at each orientation. In operation, the image receptor 116 remains in place relative to the support platform 106 while an image is taken. The immobilizer unit 104 releases the breast 102 for movement of the compression arm and tube arm 124, 126 to a different imaging orientation. For tomosynthesis, the compression arm 124 stays in place, with the breast 102 immobilized and remaining in place, while at least the tube arm 126 rotates the x-ray source 122 relative to the immobilizer unit 104 and the compressed breast 102 about the axis 128. The imaging system 100 takes plural tomosynthesis projection images of the breast 102 at respective projection angles of the beam 120 relative to the breast 102.

Concurrently and optionally, the image receptor 116 may be rotated relative to the breast support platform 106, the axis parallel to the axis 128, and in sync with the rotation of the tube arm 126. The rotation can be a proportion of the angle of the rotation of the x-ray source 122 (c.g.,) ±3.5° but may also be through a different angle selected such that the beam 120 remains substantially in the same position on the image receptor 116 for each of the plural images. The rotation can be about an axis 130, which can but need not be in the image plane of the image receptor 116. The detector rotating mechanism 118 that is coupled to the image receptor 116 can drive the image receptor 116 in a rotating motion. For tomosynthesis imaging and/or CT imaging, the breast support platform 106 can be horizontal or can be at an angle to the horizontal, e.g., at an orientation similar to that for conventional MLO imaging in mammography. The imaging system 100 can be solely a mammography system, a CT system, or solely a tomosynthesis system, other modalities such as ultrasound, or a “combo” system that can perform multiple forms of imaging. An example of a system has been offered by the assignee hereof under the trade name 3Dimensions™ system.

When the system is operated, the image receptor 116 produces imaging information in response to illumination by the imaging beam 120 and supplies it to an image processor 132 for processing and generating breast x-ray images.

A system control and workstation unit 138 including software controls the operation of the system and interacts with the operator to receive commands and deliver information including processed-ray images. The system control and workstation unit 138 may include an automatic exposure control (AEC) engine that may be configured to calculate the AEC x-ray dosage needed to perform x-ray imaging, such as a tomosynthesis sweep, on a patient's breast.

The imaging system 100 includes a floor mount or base 140 for supporting the imaging system 100 on a floor. A gantry 142 extends upwards from the floor mount 140 and rotatably supports both the tube arm 126 and the compression arm 124. The tube arm 126 and the compression arm 124 are configured to rotate discretely from each other and may also be raised and lowered along a face 144 of the gantry 142 so as to accommodate patients of different heights. The x-ray source 122 is disposed within the tube arm 126. Together, the tube arm 126 and the compression arm 124 may be referred to as a C-arm 145. In some examples, the compression arm 124 and/or the entire C-arm 145 may be configured to tilt relative to the face 144 (c.g., tilt upwards or downwards for patient positioning). In an example, the tilt angle may be between ±10° with outward tilt facilitating imaging seated persons and inward tilt facilitating imaging persons with stooped posture. The tilt axis is orthogonal to the axis 128 of the C-arm 145.

A number of interfaces and display screens are disposed on the imaging system 100. These include a foot display screen 146, a gantry interface 148, a support arm interface 150, and a compression arm interface 152. In general, the various interfaces 148, 150, and 152 may include one or more tactile buttons, knobs, switches, as well as one or more display screens, including capacitive touch screens with graphic user interfaces (GUIs) so as to enable user interaction with and control of the imaging system 100. In general, the foot display screen 146 is primarily a display screen, though a capacitive touch screen might be utilized if required or desired.

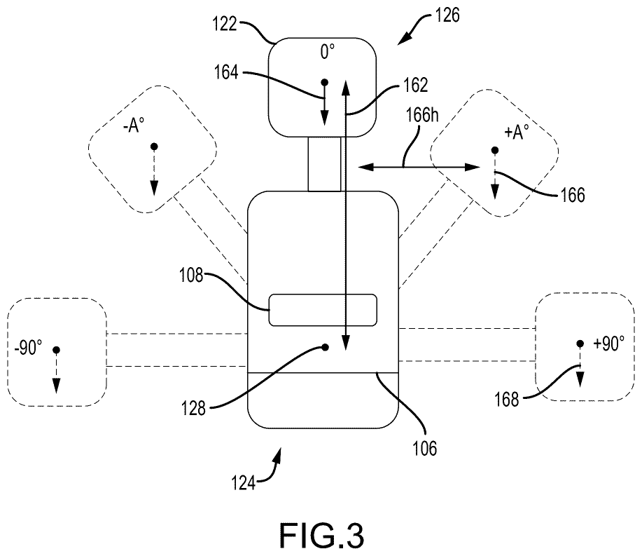

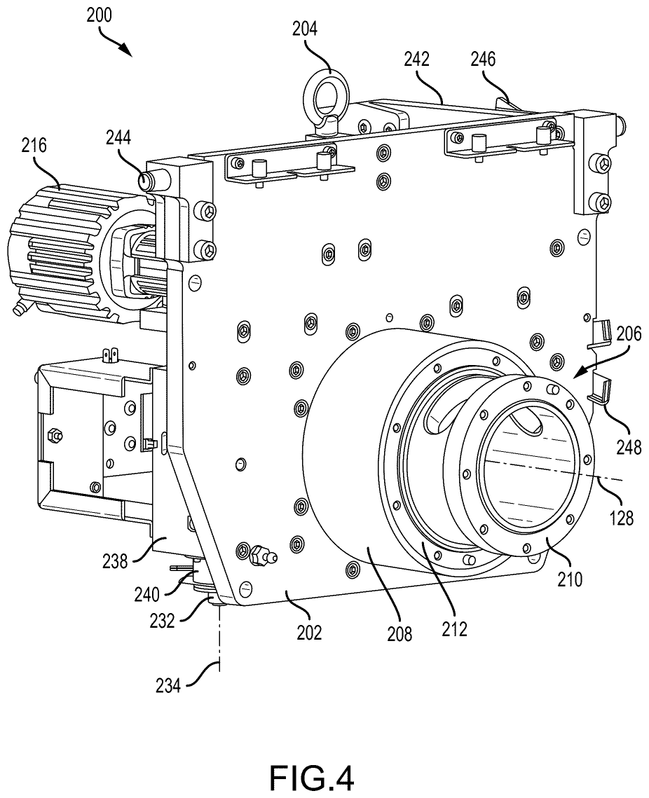

FIG. 3 is a schematic view of the compression arm 124 and the tube arm 126 of the imaging system 100 (shown in FIGS. 1 and 2). The tube arm 126 includes the x-ray source 122 that is disposed a distance 162 from the rotational axis 128. As described above, the x-ray source 122 is configured to rotate around the rotational axis 128 in order to facilitate imaging procedures. When the x-ray source 122 is positioned vertically, the tube arm 126 is at a 0° tube arm angle position. The x-ray source 122 can rotate in both a clockwise and counterclockwise direction relative to the 0° tube arm angle position, and in the example, towards a ±90° tube arm angle position. The x-ray source 122 can be positioned at any intermediate tube arm angle position ±A as required or desired. Movement of the tube arm 126 is driven by a rotational drive assembly 200 described further below in reference to FIGS. 4-6. In examples, the x-ray source 122 can rotate past the ±90° tube arm angle position as required or desired.

The x-ray source 122 has a relatively large weight that the tube arm 126 is configured to support and transfer the supported load towards the gantry 142 (shown in FIG. 2). Additionally, the system components are designed with a safety factor that further increases the loads that need to be supported. The weight of the tube arm 126 is off-center relative to the rotational axis 128. When the x-ray source 122 is at the 0° tube arm angle position, a load path 164 for the weight of the tube arm 126 is substantially vertical and aligned with the rotational axis 128. Accordingly, loading forces being transferred within the rotational drive assembly 200 are relatively low because there are few loads creating a moment arm relative to the rotational axis 128. However, when the x-ray source 122 moves out of the 0° tube arm angle position, the tube arm 126 forms a moment arm force 166 relative to the rotational axis 128 with a load path that is horizontally offset 166h from the rotational axis 128. The horizontal offset 166h induces moment arm loads within the rotational drive assembly 200 that the components within are configured to support. A maximum load 168 condition is when the x-ray source 122 is at the ±90° tube arm angle position as the load path is horizontally spaced the furthest from the rotational axis 128 and at the entire distance 162. During movement of the tube arm 126, the loading forces through the rotational drive assembly 200 are not uniform.

The compression arm 124 is also rotatable around the rotational axis 128. The compression arm 124 includes the paddle 108, the support platform 106, and other components. As such, the compression arm 124 also induces loading forces through the rotational drive assembly 200 and similar to the forces described above because the weight of the compression arm 124 is not centered with the rotational axis 128. In aspects, the loading forces induced by the compression arm 124 are smaller than those induced by the tube arm 126.

Rotational movement of the x-ray source 122 and/or compression arm 124 are driven by the imaging mode being used. Mammography imaging modes can include a craniocaudal (CC) view that is performed with the x-ray source 122 at the 0° tube arm angle position, one or more mediolateral oblique (MLO) views that is performed with the x-ray source 122 between 30° and 60° on the gantry with the compression arm 124 positioned 90° relative to the x-ray source 122, and other image types are also contemplated herein. Tomosynthesis imaging modes can include a sweep of the x-ray source 122 between ±7.5°, wide angle tomosynthesis with a sweep of the x-ray source 122 up to and including ±30°, and other angular positions are also contemplated herein. In examples, tomo sweeps can occur from the CC or the MLO 0° imaging angle starting position as required or desired. Other imaging modes are also contemplated herein such as stereotactic imaging, wide angle imaging, CT imaging, lateral imaging, etc. As described above, when the tube arm 126 is moved out of the 0° tube arm angle position, the configuration of its rotational support structure is known to result in an angular discontinuity jump due to the weight of the tube arm 126 and this jump can produce undesirable image artifacts.

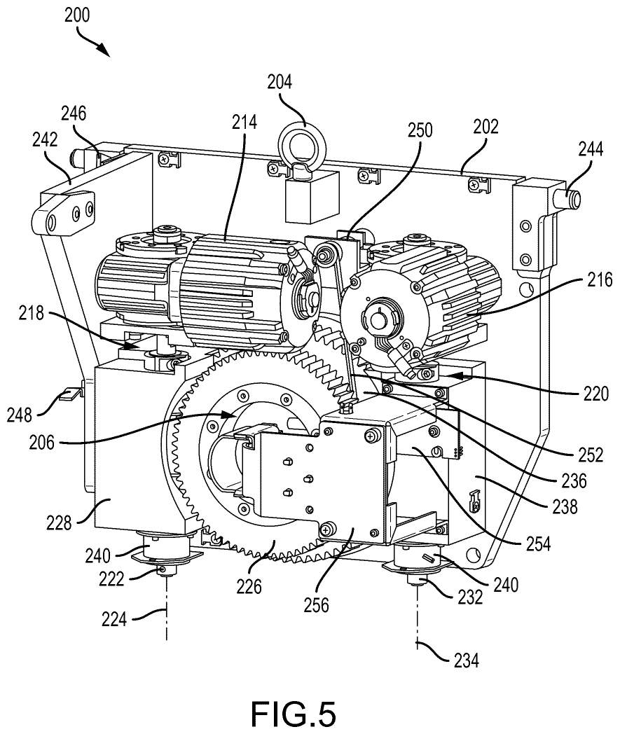

FIG. 4 is a perspective view of a rotational drive assembly 200 for rotation of the compression arm 124 and the tube arm 126 (shown in FIG. 3). FIG. 5 is another perspective view of the rotational drive assembly 200. Referring concurrently to FIGS. 4 and 5, the rotational drive assembly 200 is configured to be mounted at least partially within the gantry 142 (shown in FIG. 2) and drive rotational movement of the compression arm 124 and the tube arm 126 as described herein. The rotational drive assembly 200 includes a frame 202 for coupling the rotational drive assembly 200 within the gantry 142. The frame 202 includes an eyebolt 204 for facilitating mounting the rotational drive assembly 200 within the gantry 142.

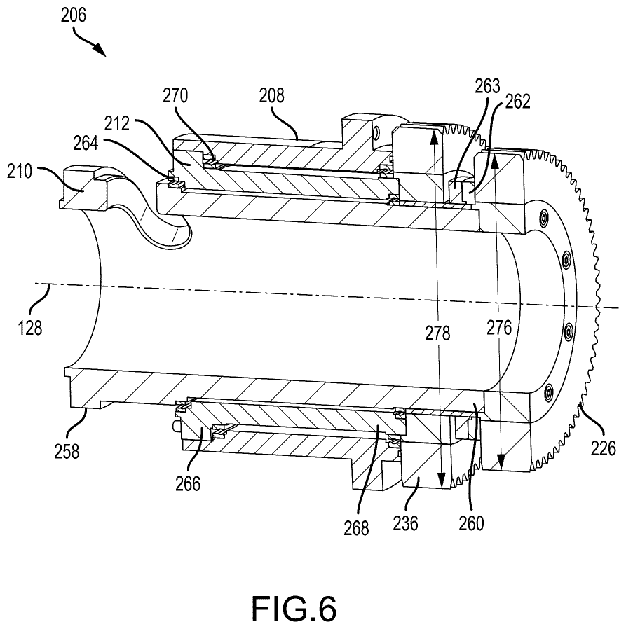

A tube shaft assembly 206 is rotatably supported on the frame 202 at a hub 208. The tube shaft assembly 206 includes a compression arm tube shaft 210 that is configured to support the compression arm 124 at a distal end and facilitate rotation thereof around the axis 128. The tube shaft assembly 206 also includes a tube arm tube shaft 212 that is configured to support the tube arm 126 at a distal end and facilitate rotation thereof around the axis 128. The tube shaft assembly 206 is described further below in reference to FIG. 6.

The rotational drive assembly 200 includes a compression arm actuator 214 configured to selectively drive rotation of the compression arm tube shaft 210 and a tube arm actuator 216 configured to selectively drive rotation of the tube arm tube shaft 212. The actuators 214, 216 are supported on the frame 202 and may be any type of actuator that facilitates driving rotational movement of the tube shaft assembly 206. In the example, the actuators 214, 216 are electrical motors that can drive rotation in two directions, a first rotational direction and an opposite second rotational direction.

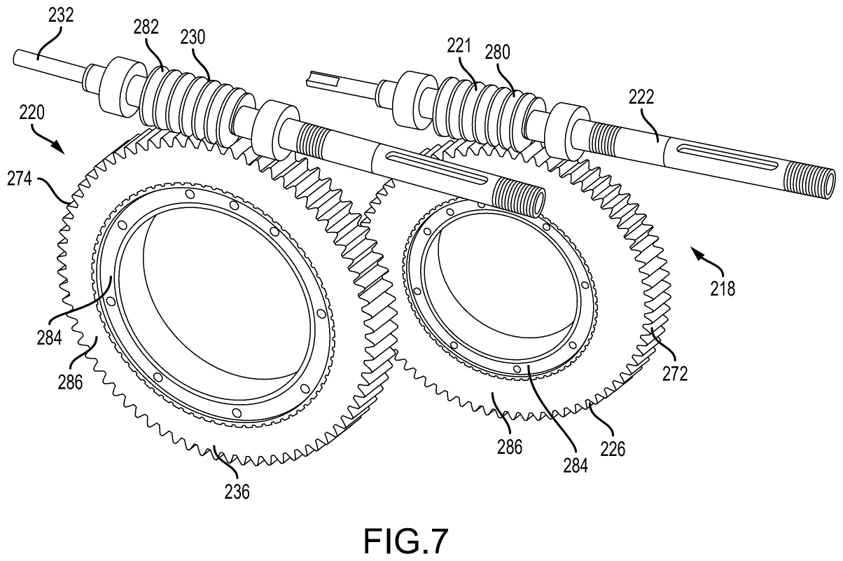

A compression arm gearset 218 operationally couples the compression arm actuator 214 to the compression arm tube shaft 210 and a tube arm gearset 220 operationally coupled the tube arm actuator 216 to the tube arm tube shaft 212. The compression arm gearset 218 includes a first driving gear 221 (shown in FIG. 7) coupled to a first drive shaft 222 that is rotatable around a first drive axis 224 and a first driven gear 226 that is coupled to a proximal end of the compression arm tube shaft 210. In the example, the first driving gear 221 is supported in a first housing 228 coupled to the frame 202. The tube arm gearset 220 similarly includes a second driving gear 230 (shown in FIG. 7) coupled a second drive shaft 232 that is rotatable around a second drive axis 234 and a second driven gear 236 that is coupled to a proximal end of the tube arm tube shaft 212. The second driving gear 230 is supported in a second housing 238 coupled to the frame 202. In the example, the first and second drive axes 224, 234 are parallel to each other, and are orthogonal to the rotational axis 128 of the rotational drive assembly 200. Brakes 240 are also engaged with each of the drive shafts 222, 232.

The rotational drive assembly 200 also includes a tilt arm plate 242 that facilitates the rotational drive assembly 200 tilting about pivot pins 244 on the frame 202 and relative to the face of the gantry 142 via a tilt angle encoder drive gear 246. The actuation mechanisms for the tilting motion are not shown. One or more tilt limit switch actuation plates 248 are coupled to the tilt arm plate 242. An analog encoder 250 is coupled to the frame 202 and engaged with the compression arm portion of the tube shaft assembly 206 via a timing belt 252. The analog encoder 250 is configured to measure rotational position of the compression arm portion of the tube shaft assembly 206. A compression arm rotation limit switch circuit board 254 prevents driving the compression arm rotation beyond its safe mechanical limits, and a cable guide 256 facilitates cable management though the rotational drive assembly 200 and communication with the compression arm 124 and the tube arm 126.

FIG. 6 is a cross-sectional, perspective view of the tube shaft assembly 206 of the rotational drive assembly 200 (shown in FIGS. 4-5). The tube shaft assembly 206 includes the compression arm tube shaft 210 and the tube arm tube shaft 212, the shafts 210, 212 are independently rotatable around the rotational axis 128. The compression arm tube shaft 210 has a distal end 258 configured to support the compression arm 124 (shown in FIGS. 1 and 2) and an opposite proximal end 260 that the first driven gear 226 is coupled to. A pulley 262 and a cam 263 are disposed adjacent to the first driven gear 226 and couples the timing belt 252 to the encoder 250, while the cam 263 actuates the limit switch on PCB 254 (shown in FIG. 5).

The tube arm tube shaft 212 is rotationally supported by a pair of first bearings 264 on the compression arm tube shaft 210 and positioned radially outside. A distal end 266 of the tube arm tube shaft 212 is configured to support the tube arm 126 (shown in FIGS. 1 and 2) and an opposite proximal end 268 that the second driven gear 236 is coupled to. In the example, the tube arm tube shaft 212 has an axial length that is shorter than the compression arm tube shaft 210. The tube arm tube shaft 212 is rotationally supported by a pair of second bearings 270 within the hub 208 that statically mounts to the frame 202 of the rotational drive assembly 200.

The compression arm tube shaft 210, the first driven gear 226, the tube arm tube shaft 212, and the second driven gear 236 are all coaxial relative to the rotational axis 128. In the example, the first driven gear 226 has a plurality of first teeth 272 (shown in FIG. 7) and the second driven gear 236 has a plurality of second teeth 274 (shown in FIG. 7). The teeth 272, 274 are only illustrated schematically in FIG. 5. An outer diameter 276 of the first teeth 272 of the first driven gear 226 is smaller than an outer diameter 278 of the second teeth 274 of the second driven gear 236. In an example, the first teeth 272 have a 70-tooth count with a 7 inch pitch diameter, and the second teeth 274 have an 80-tooth count with an 8 inch pitch diameter. In other examples, the outer diameter, tooth count, and pitch diameter may be different than the above listed configurations as required or desired.

FIG. 7 is a perspective view of the compression arm gearset 218 and the tube arm gearset 220 of the rotational drive assembly 200 (shown in FIGS. 4-5). In the example, the first driving gear 221 is meshed with the first driven gear 226 so that rotation of the first driving gear 221 drives rotation of the first driven gear 226. The second driving gear 230 is meshed with the second driven gear 236 so that rotation of the second driving gear 230 drives rotation of the second driven gear 236. In the example, the first driving gear 221 is a wormshaft with at least one first tooth 280 and the second driving gear 230 is a wormshaft with at least one second tooth 282. The at least one first and second teeth 280, 282 are helical teeth. The first and second driving gears 221, 230 are formed from a metal material. In an aspect, the first driving gear 221 and the second driving gear 230 have identical tooth geometries.

In operation, the gearsets 218, 220 facilitate rotational movement of the compression arm 124 and the tube arm 126 (both shown in FIG. 3) and via drive from the actuators. As described above, both the compression arm 124 and the tube arm 126 are heavy and can generate large off-center loads relative to the rotational axis 128 (shown in FIG. 6). For example, the compression arm 124 carries the x-ray detector components and the paddle drive components, and the tube arm 126 carries the x-ray source components that are disposed at a large radial distance from the rotational axis 128. Additionally, while rotational speed of the compression arm 124 and the tube arm 126 need not be fast, accurate angular positioning is desired so as to facilitate the imaging procedures such as mammography, tomosynthesis, and the like. Accordingly, using the worm gearing described herein provides for a low-speed, high-torque drive system that enables rotation in both rotational directions and is robust, quiet, and self-locking and/or resistance to back driving. It is appreciated that the rotational drive assembly gearsets may use other types of gears as required or desired, and such as, pinion gears, ring and pinion gears, etc.

In the example, both the first and second driven gears 226, 236 are composite gears with a cylindrical metal core 284 and a polymer outer layer 286 forming the respective teeth 272, 274. In an aspect, the polymer outer layer 286 is cast on to the metal core 284, and for example, overmolded to a knurled steel core. In one example, the polymer outer layer 286 is nylon 12. It is appreciated that other polymer materials may alternatively be used for the composite gears that are described herein. The alternative polymer materials may have the same or similar material properties to that of nylon 12. In aspects, a radial thickness of the polymer outer layer 286 is greater than a radial thickness of the metal core 284. In other aspects, the radial thickness of the polymer outer layer 286 is at least twice, three times, four times, or more the radial thickness of the metal core 284. The polymer thickness and/or width may be based on speed and/or load bearing capacities of the gearing system.

When compared to all metal gearing, use of polymer on the first and second driven gears 226, 236 provide a number of benefits. Friction between gears is reduced, thereby increasing gearset efficiencies. Torque transmission is increased, while noise, vibration, and/or heat generation are reduced during rotational operation. The first and second driven gears 226, 236 described herein are also operable without maintenance lubrication thereby simplifying the rotational drive assembly for assembly and service. Wear is reduced or measurably eliminated during the operational lifetime of the gearsets and testing has demonstrated measurable wear is prevented of a rotational lifetime of 27 years, three times the nominal lifetime of such systems. In some examples, a one-time break in lubrication is applied to the gearsets prior to initial operation, but no further lubrication need be applied during the operational lifetime of the gearsets.

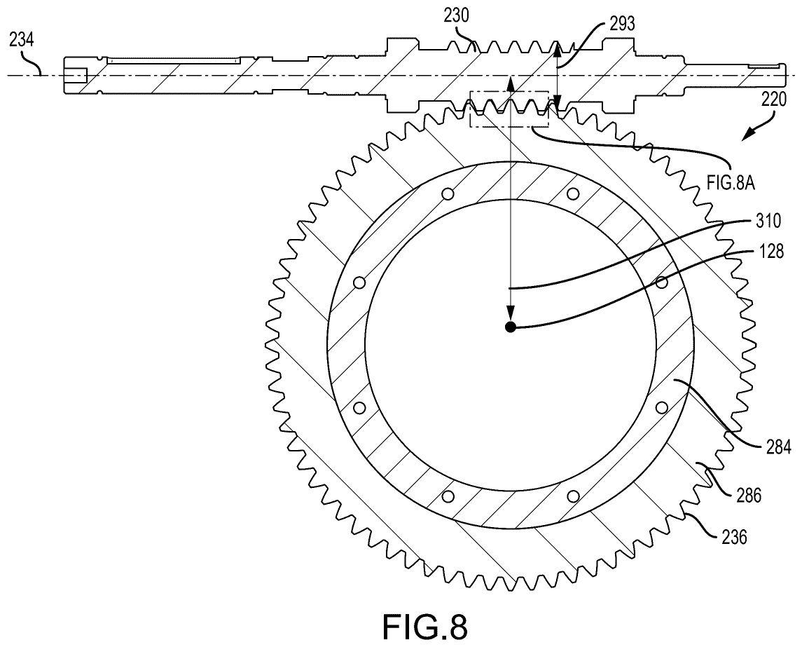

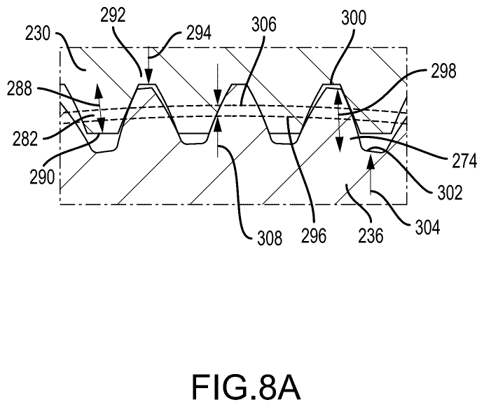

FIG. 8 is a schematic view of the tube arm gearset 220 and FIG. 8A is a detailed view of the gearset mesh. Referring to FIGS. 8 and 8A, while the tube arm gearset 220 is described below, it is appreciated that the compression arm gearset 218 (shown in FIG. 7) is similarly designed. The tube arm gearset 220 includes the driving gear 230 that is configured to rotate around the drive axis 234 and the driven gear 236 that is configured to rotate around the rotational axis 128 (e.g., in and out of the page). The driving gear 230 is a wormshaft with a helical tooth 282. The tooth 282 has a tooth depth 288 defined from a distal end 290 to a hub 292 of the gear 230. An outer diameter 293 of the driving gear 230 is measured to the distal end 290 of the tooth 282, while an inner diameter 294 of the driving gear 230 is measured at the hub 292. The tooth 282 also includes a tooth thickness, measured orthogonal to the depth 288 and that tapers inwardly towards the distal end 290, and a tooth pitch, measured circumferentially between the distal ends. A pitch radius 296 is defined from the drive axis 234 to the location on the tooth 282 that engages with the driven gear 236 in a theoretical meshed condition. In the example, the pitch radius 296 is disposed between the hub 292 and the distal end 290 of the tooth 282.

The driven gear 236 is a wormgear with a plurality of teeth 274. The teeth 274 each have a tooth depth 298 defined from a distal end 300 to a hub 302 of the gear 236. The outer diameter 278 (shown in FIG. 6) is measured to the distal end 300 of the teeth 274, while an inner diameter 304 of the driven gear 236 is measured at the hub 302. The teeth 274 also includes a tooth thickness, measured orthogonal to the depth 298 and that tapers inwardly towards the distal end 290, and a tooth pitch, measured circumferentially between the distal ends. A pitch radius 306 is defined from the rotational axis 128 to the location on the teeth 274 that engages with the driving gear 230 in a theoretical meshed condition. In the example, the pitch radius 306 is disposed between the hub 302 and the distal end 300 of the teeth 274.

Theoretically, aligning the pitch radius 296 of the driving gear 230 and the pitch radius 306 of the driven gear 236 places the gears 230, 236 in a fully meshed condition for operation. Fully-meshed is used herein to describe the pitch radius 296 touching the pitch radius 306 at a tangential line to both circles. In practice, however, the gears 230, 236 are under-meshed such that clearance is provided due to manufacturing tolerances and lubrication. Under-meshed is used herein to describe the pitch radius 296 spaced apart from the pitch radius 306 thereby providing clearance to the gear teeth. Under-meshing the driving gear 230 and the driven gear 236 described herein, however, results in an angular position jump when the tube arm 126 (shown in FIG. 3) rotates out of the 0° tube arm angle position due to the loading conditions. This angular position jump due to movement loading on the gearset 220 can be referred to as backlash or lash and that is the clearance between the gear teeth 274, 282 as described further above.

Accordingly, in the example, the gears 230, 236 are over-meshed such that at least some of the engaged teeth 274 of the driven gear 236 are forced in direct contact against the driving gear 230. In examples, this direct contact of the engaged teeth 274 against the driving gear 230 induces a loading force within the engaged teeth 274 that acts against the tooth 282 of the driving gear 230 and restricts or prevents an angular position jump when the tube arm 126 rotates out of the 0° tube arm angle position. The loading force within the engaged teeth 274 at least partially counteracts the weight of the tube arm 126 that is being transferred through the gearset 220 as described above and so that lash does not occur. Over-meshing the gearset 220 is enabled because of the driven gear 236 having teeth 274 formed out of polymer material as described above and with material properties that enable elastic loading of the teeth 274. It should be appreciated that over-meshing has an upper limit, whereby the loading force within the engaged teeth 274 is large enough to bind the gears 230, 236 and restrict or prevent rotational operation of the gearset 220 or load the polymer teeth into an inelastic (e.g., plastic) zone. As such, the over-meshed configuration needs generate a loading force that is large enough to prevent or reduce lash, while also small enough not to bind the gears 230, 236 or inelastically load the polymer teeth.

In an example, an over-mesh configuration of the gears may be defined by the pitch radius 296 of the driving gear 230 overlapping 308 the pitch radius 306 of the driven gear 236 such that the gear teeth 274, 282 have an interference fit. As such, a distance 310 between the drive axis 234 and the rotational axis 128 is less than the pitch radius 296 and the pitch radius 306 added together and adjusting the distance 310 at least partially determines the loading force within the driven gear 236. By reducing the distance 310 between the drive axis 234 and the rotational axis 128, the teeth 274 of the driven gear 236 at least partially compress and or bend against the tooth 282 of the driving gear 230 thereby inducing a loading force within the teeth 274.

In an aspect, the overlap 308 of the pitch radii 296, 306 is between 1% and 20% of the distance 310 between the drive axis 234 and the rotational axis 128 in a fully-meshed configuration. The overlap 308 of the pitch radii 296, 306 may be between 1% and 10% of the distance 310 between the drive axis 234 and the rotational axis 128 in a fully-meshed configuration. The overlap 308 of the pitch radii 296, 306 may be between 1% and 5% of the distance 310 between the drive axis 234 and the rotational axis 128 in a fully-meshed configuration.

In another example, an over-mesh configuration may be defined by the deflection of the teeth 274 of the driven gear 236 when engaged with the tooth 282 of the driving gear 230 in the interference fit. The deflection of the teeth 274 may include both compression and bending stresses induced in the teeth and adjusting the compression and bending stresses at least partially determines the loading force within the driven gear 236. Compression and bending stresses can be set by the distance 310 between gears as described above. Additionally, or alternatively, the shape and/or size of the teeth 274 can be used to determine the compression and bending stresses induced within the teeth 274 of the driven gear 236. It is appreciated, that the shape and/or size of the driving gear teeth may additionally or alternatively be used to form the compression and bending stresses.

In an aspect, the loading force of the teeth 274 of the driven gear 236 is between 0.1 kilopound per square inch (ksi) and 12 ksi. The loading force of the teeth 274 of the driven gear 236 may be between 0.1 ksi and 9 ksi. The loading force of the teeth 274 of the driven gear 236 may be between 0.1 ksi and 5 ksi. The loading force of the teeth 274 of the driven gear is set when the tube arm 126 is at a 0° tube arm angle position.

As described above, the gear loading through the gearset 220 is not uniform. Larger loads are transferred through the gearset 220 as the tube arm 126 rotates closer to the ±90° tube arm angle position. As such, in one example, over-meshing the gears 230, 236 may be configured to reduce or prevent lash though the entire loading cycle of the gearset 220 as the tube arm 126 rotates toward the ±90° tube arm angle position. In this example, the maximum loading condition when the tube arm 126 is at the ±90° tube arm angle position is determined and the corresponding loading force is induced within the driven gear 236, thereby inducing a positive loading force within the gears that is greater than the load of the tube arm 126.

In other examples, however, it may not be desirable to induce such a large loading force within the driven gear. For example, such a loading force may exceed the elastic properties of the polymer material, motor power may not be sufficient to overcome such a loading force, such a loading force may cause the gears to at least partially bind, or such a loading force may induce over lash (e.g., further movement of the tube arm) when the tube arm is closer to the unloaded 0° tube arm angle position. Additionally, increasing movement to bring the gears closer together results in compression of the composite gear in the region of contact on both sides of the wormshaft, leading to increasing contact areas on the adjacent wormgear teeth, and limiting the loading force induced therein. As such, in other examples, over-meshing of the gears is configured so that lash is reduced or prevented for tube arm angle positions that are less than ±90°. In an aspect, lash in the gears is reduced or prevented for a ±60° tube arm angle position, a ±30° tube arm angle position, a ±15° tube arm angle position, a ±10° tube arm angle position, a ±7.5 tube arm angle position, a ±5° tube arm angle position, or a ±3° tube arm angle position.

By reducing the tube arm angles that lash is restricted or reduced, the outside heavy loading conditions do not apply heavy loading stresses on the gear teeth and lash at the outer positions that does occur can be more easily accounted for by load position encoders within the imaging system. In these examples, lash is reduced or prevented at a predefined subset of tube arm angle positions around the 0° position so as to reduce wear on the system and to increase movement performance, but at the other positions, lash may occur. The tube arm angles that lash is restricted or reduced may be referred to as a zero lash region. Once outside of the zero-lash region, the loading force within the composite gear is configured to allow lash movement of the tube arm, but this lash movement may be reduced as compared to what would otherwise be present.

Determining the loading force or pitch radius overlap for the required or desired zero lash region can be performed in different ways. In one example, a force-displacement approach can be utilized. The steel worm gear is fixed and a calculated torque load or an equivalent tangential force is applied to the composite gear to measure tooth displacement at the pitch circle. The applied load can be based on empirically derived data and interpolated values for loads and lash (c.g., displacement) at tube arm angles from 0-90°. Using this information, an initial lash setting at a given force can be selected.

In another example, a pressure-based approach can be utilized. Tooth contact area can be characterized as the tooth pressure builds from zero to a maximum (e.g., at the high angle lash region) and this enables assembly of the magnitude of tooth pressure under both loaded and zero lash configurations. Unit pressure scales fairly linearly from zero to a maximum, and as such, a relative large lash-setting tangential force can be applied so that the information can be used to set the pressure for the required or desired zero lash region. Tooth pressure may be measured, for example, by painting the wormshaft with a machinist's dye, engaging a meshing force, and observing the resulting mesh area on the wormgear tecth.

In still another example, a sensing-based approach can be utilized. The rotational movement of the wormgear is sensed as a desired tangential test force is reached. By measuring tooth displacement, an increasing tangential force is applied to the wormgear tooth. The tangential force is relatively small, and the information can be used to set the pre-load forces in the composite gear. An illustrative method to set zero-lash is to apply a criterion tangential force to any wormgear tooth, and adjust the pitch center distance such that tangential movement of that or another tooth is just observed with a sensitive instrument. For example, setting ±7.5° zero lash will load a gear tooth to 35 pounds force, while observing a maximum tangential tooth displacement of 0.0005 inches.

The foregoing discussions and setting methods pertain to the larger 80-tooth tube arm gear, with an 8.0-inch pitch diameter. The compression arm gear is smaller, with 70 teeth and a 7.0-inch pitch diameter. Since the worm gear teeth can be the same size and shape on both gears, the tangential force to deflect them needs to be the same. Since the constant tangential force is applied at a smaller action radius, the equivalent torque the gear experiences is less, but not the tangential force. Additionally, composite gearing as described herein may be utilized in other drive components of the imaging system. For example, a paddle drive is disposed within the compression arm and is configured to move the paddle. The paddle drive typically includes a leadscrew and nut in combination with spur gearing driving linear movement of the paddle. The nut may be formed as a composite element so as to reduce lash within this type of system.

The determined loading of the composite gear for overmeshing can be implemented by either pushing the wormgear tooth with the corresponding tangential force or torquing the wormgear to achieve the same tangential force.

The over-mesh configuration may be measured and set in the field, or on an assembled machine, through reporting the angular position of the tube arm. For example, the angular position of the tube arm is measured and reported with a resolution to 0.01°. A spring scale capable of measuring up to a 5-pound force is provided. The tube arm and the compression arm are moved to the 0° angular position. Applying a 4.5-pound force in both directions on the tube arm and determining that the total movement is 0.01° or less. If setting is required, positional adjustment of the driven gear can be made. These steps are repeated for the compression arm with a 5-pound force and so that the total movement is 0.01° or less.

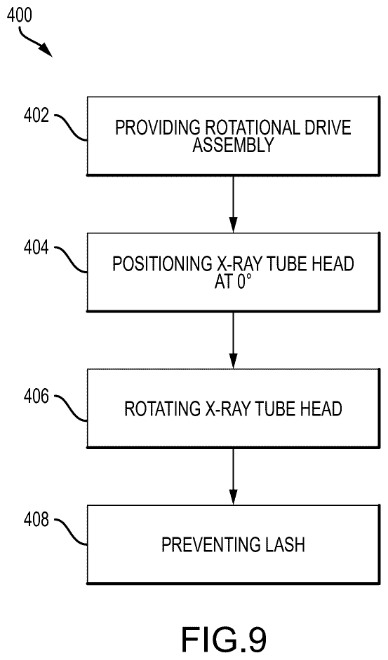

FIG. 9 is a flowchart illustrating an exemplary method 400 of rotating an x-ray tube arm of an imaging system. The example methods and operations can be implemented or performed by the assemblies described herein (e.g., the imaging system 100 and rotational drive assembly 200 shown in FIGS. 1-8). The imaging system includes a gantry rotationally supporting the x-ray tube arm and an arm having an immobilization system including a paddle, a support platform, and an x-ray receptor disposed below the support platform.

The method 400 begins with providing a rotational drive assembly (operation 402). The rotational drive assembly includes a tube shaft rotatable around a tube arm axis and supporting the x-ray tube arm, an actuator, and a gearset. The gearset includes a driving gear coupled to the actuator and having at least one first tooth and a driven gear coupled to the tube shaft and having at least one second tooth. The at least one second tooth of the driven gear is formed from a polymer material, and the driven gear is over-meshed with the driving gear such that a loading force is induced within the at least one second tooth. The x-ray tube including an x-ray source is positioned at a 0° tube arm angle relative to the gantry via the rotational drive assembly (operation 404).

The x-ray tube arm is rotated about the tube arm axis and out of the 0° tube arm angle by actuating the driving gear thereby rotating the driven gear around the tube arm axis (operation 406). Based on the loading force within the at least one second tooth, lash of the x-ray tube arm assembly is prevented via the driven gear when a weight of the x-ray tube arm assembly moves out of the 0° tube arm angle (operation 408). In examples, rotation of the x-ray tube arm is part of an imaging mode, such as tomosynthesis, so that image artifacts are reduced or eliminated in the corresponding images. The loading force may be generated based on a force-displacement approach, a pressure approach, a sensing approach (all described above), or any other setting that is developed in the future.

In examples, during rotation of the tube arm there is a continuous and seamless transition from the overmeshed gear teeth condition at zero/low/medium loading to the undermeshed gear teeth condition at higher loads. This transition can be calculated and adjusted to provide relatively uniform and consistent tooth unit pressures from zero to maximum tube arm and compression arm loadings (c.g., from 0° to) ±90°. Empirical testing has validated these computed load pressures, and even when the gearset is highly overmeshed, safe gear tooth pressures are obtained over the full range of possible motion such as with the tube arm near the machine base at 180° (e.g., the transport position of mobile installations). This uniform tooth loading system has the benefit of reducing or eliminating motion discontinuity during loading from the 0° position. Additionally, another benefit is making the drive motor loading more uniform, thereby facilitating driving the C-arm subsystems at a consistent speed. Accordingly, more uniform motion control, reduced motion artifacts, and/or reduced drivetrain noise are all facilitated and provide improvements to the known motion controllers that use nested control loops and algorithms.

### EXAMPLES

Illustrative examples of the systems and methods described herein are provided below. An embodiment of the system or method described herein may include any one or more, and any combination of, the clauses described below:

Clause 1. An x-ray imaging system including: a gantry including a rotational drive assembly; an x-ray tube arm including an x-ray source coupled to the gantry at the rotational drive assembly, the x-ray tube arm configured to selectively rotate relative to the gantry around a rotational axis via the rotational drive assembly; and an arm including an immobilization system coupled to the gantry and independently rotatable relative to the gantry around the rotational axis via the rotational axis, wherein the immobilization system includes a paddle, a support platform, and an x-ray receptor disposed below the support platform, wherein the rotational drive assembly includes: a tube shaft rotatable around the rotational axis and supporting the x-ray tube arm; an actuator configured to drive rotation of the tube shaft; and a gearset including a driving gear coupled to the actuator and having at least one first tooth and a driven gear coupled to the tube shaft and having at least one second tooth, wherein the at least one second tooth of the driven gear is formed from a polymer material, and wherein the driven gear is over-meshed with the driving gear such that the at least one second tooth is in direct contact with the at least one first tooth with the at least one second tooth at least partially deflected causing a loading force within the at least one second tooth.

Clause 2. The x-ray imaging system of any one of clauses 1-28, wherein the deflection of the at least one second tooth includes bending and compression of the at least one second tooth.

Clause 3. The x-ray imaging system of any one of clauses 1-28, wherein the loading force is between 0.1 kilopound per square inch (ksi) and 5 ksi.

Clause 4. The x-ray imaging system of any one of clauses 1-28, wherein the loading force is measured when the x-ray tube arm is at a 0° tube arm angle.

Clause 5. The x-ray imaging system of any one of clauses 1-28, wherein the loading force occurs when the x-ray tube arm is between at least a ±5° and a ±30° tube arm angles.

Clause 6. The x-ray imaging system of any one of clauses 1-28, wherein the driving gear is formed from a metal material, the driving gear is a wormshaft and the at least one first tooth is a helical tooth.

Clause 7. The x-ray imaging system of any one of clauses 1-28, wherein the driving gear includes a metal core, the polymer material cast on the metal core and forming a tooth ring.

Clause 8. The x-ray imaging system of any one of clauses 1-28, wherein the rotational drive assembly includes a second tube shaft supporting the arm, a second actuator configured to drive rotation of the second tube shaft, and a second gearset, the second gearset having a smaller diameter driven gear than the driven gear of the tube shaft of the x-ray tube arm.

Clause 9. The x-ray imaging system of any one of clauses 1-28, wherein the driven gear is rotatable around the rotational axis and the driving gear is rotatable around a drive axis, and wherein a distance between the drive axis and the rotational axis is such that the driving gear has an interference fit with the driven gear and a first pitch radius of the at least one first tooth at least partially overlaps with a second pitch radius of the at least one second tooth.

Clause 10. The x-ray imaging system of any one of clauses 1-28, wherein the overlap of the first pitch radius of the at least one first tooth and the second pitch radius of the at least one second tooth compresses the at least one second tooth inducing the loading force therein.

Clause 11. The x-ray imaging system of any one of clauses 1-28, wherein the overlap of the first pitch radius of the at least one first tooth and the second pitch radius of the at least one second tooth is between 1% and 5% of the distance between the drive axis and the rotational axis at a 0° tube arm angle in a fully-meshed configuration.

Clause 12. The x-ray imaging system of any one of clauses 1-28, wherein the interference fit is measured when the x-ray tube arm is at a 0° tube arm angle.

Clause 13. The x-ray imaging system of any one of clauses 1-28, wherein the interference fit occurs when the x-ray tube arm is between at least a ±5° and a ±30° tube arm angles.

Clause 14. The x-ray imaging system of any one of clauses 1-28, wherein the driving gear is formed from a metal material, the driving gear is a wormshaft and the at least one first tooth is a helical tooth.

Clause 15. The x-ray imaging system of any one of clauses 1-28, wherein the driving gear includes a metal core, the polymer material cast on the metal core and forming a tooth ring.

Clause 16. The x-ray imaging system of any one of clauses 1-28, wherein the rotational drive assembly includes a second tube shaft supporting the arm, a second actuator configured to drive rotation of the second tube shaft, and a second gearset, the second gearset having a smaller diameter driven gear than the driven gear of the tube shaft of the x-ray tube arm.

Clause 17. A method of rotating an x-ray tube arm of an imaging system, the imaging system including a gantry rotationally supporting the x-ray tube arm and an arm having an immobilization system including a paddle, a support platform, and an x-ray receptor disposed below the support platform, the method including: providing a rotational drive assembly, wherein the rotational drive assembly includes a tube shaft rotatable around a tube arm axis and supporting the x-ray tube arm, an actuator, and a gearset including a driving gear coupled to the actuator and having at least one first tooth and a driven gear coupled to the tube shaft and having at least one second tooth, wherein the at least one second tooth of the driven gear is formed from a polymer material, and wherein the driven gear is over-meshed with the driving gear such that a loading force is induced within the at least one second tooth; positioning, via the rotational drive assembly, the x-ray tube arm that includes an x-ray source at a 0° tube arm angle relative to the gantry; rotating the x-ray tube arm about the tube arm axis and out of the 0° tube arm angle by actuating the driving gear thereby rotating the driven gear around the tube arm axis; and based on the loading force within the at least one second tooth, preventing lash of the x-ray tube arm via the driven gear when a weight of the x-ray tube arm moves out of the 0° tube arm angle.

Clause 18. The method of any one of clauses 1-28, further including inducing the loading force based on a force-displacement approach, a pressure approach, or a sensing approach.

Clause 19. The method of any one of clauses 1-28, wherein rotating the x-ray tube arm occurs during a tomosynthesis imaging mode of the imaging system.

Clause 20. The method of any one of clauses 1-28, wherein the loading force prevents lash of the x-ray tube arm at least between a ±5° and a ±30° tube arm angles.

Clause 21. An x-ray imaging system including: a gantry including a rotational drive assembly defining a rotational axis; an x-ray tube arm including an x-ray source coupled to the gantry at the rotational drive assembly, the x-ray tube arm configured to selectively rotate relative to the gantry via the rotational drive assembly; and an arm including an immobilization system coupled to the gantry at the rotational drive assembly and independently rotatable relative to the gantry, wherein the immobilization system includes a paddle, a support platform, and an x-ray receptor disposed below the support platform, wherein the rotational drive assembly includes: a tube shaft rotatable around the rotational axis and supporting the x-ray tube arm; an actuator configured to drive rotation of the tube shaft; and a gearset including a driving gear coupled to the actuator and having at least one first tooth and a driven gear coupled to the tube shaft, rotatable around the rotational axis, and having at least one second tooth, wherein the at least one second tooth of the driven gear is formed from a polymer material, wherein the driving gear is rotatable around a drive axis, and wherein a distance between the drive axis and the rotational axis is such that the driving gear has an interference fit with the driven gear and a first pitch radius of the at least one first tooth at least partially overlaps with a second pitch radius of the at least one second tooth.

Clause 22. The x-ray imaging system of any one of clauses 1-28, wherein the overlap of the first pitch radius of the at least one first tooth and the second pitch radius of the at least one first tooth compresses the at least one second tooth inducing a loading force therein.

Clause 23. The x-ray imaging system of any one of clauses 1-28, wherein the overlap of the first pitch radius of the at least one first tooth and the second pitch radius of the at least one second tooth is between 1% and 5% of the distance between the drive axis and the rotational axis at a 0° tube arm angle in a fully-meshed configuration.

Clause 24. The x-ray imaging system of any one of clauses 1-28, wherein the interference fit is measured when the x-ray tube arm is at a 0° tube arm angle.

Clause 25. The x-ray imaging system of any one of clauses 1-28, wherein the interference fit occurs when the x-ray tube arm is between at least a ±5° and a ±30° tube arm angles.

Clause 26. The x-ray imaging system of any one of clauses 1-28, wherein the driving gear is formed from a metal material, the driving gear is a wormshaft and the at least one first tooth is a helical tooth.

Clause 27. The x-ray imaging system of any one of clauses 1-28, wherein the driving gear includes a metal core, the polymer material cast on the metal core and forming a tooth ring.

Clause 28. The x-ray imaging system of any one of clauses 1-28, wherein the rotational drive assembly includes a second tube shaft supporting the arm, a second actuator configured to drive rotation of the second tube shaft, and a second gearset, the second gearset having a smaller diameter driven gear than the driven gear of the tube shaft of the x-ray tube arm.

This disclosure described some examples of the present technology with reference to the accompanying drawings, in which only some of the possible examples were shown. Other aspects can, however, be embodied in many different forms and should not be construed as limited to the examples set forth herein. Rather, these examples were provided so that this disclosure was thorough and complete and fully conveyed the scope of the possible examples to those skilled in the art.

Although specific examples were described herein, the scope of the technology is not limited to those specific examples. One skilled in the art will recognize other examples or improvements that are within the scope of the present technology. Therefore, the specific structure, acts, or media are disclosed only as illustrative examples. Examples according to the technology may also combine elements or components of those that are disclosed in general but not expressly exemplified in combination, unless otherwise stated herein. The scope of the technology is defined by the following claims and any equivalents therein.

What is claimed is:

1. An x-ray imaging system comprising:
a gantry including a rotational drive assembly;
an x-ray tube arm including an x-ray source coupled to the gantry at the rotational drive assembly, the x-ray tube arm configured to selectively rotate relative to the gantry around a rotational axis via the rotational drive assembly; and
an arm including an immobilization system coupled to the gantry and independently rotatable relative to the gantry around the rotational axis via the rotational axis, wherein the immobilization system includes a paddle, a support platform, and an x-ray receptor disposed below the support platform,
wherein the rotational drive assembly includes:
a tube shaft rotatable around the rotational axis and supporting the x-ray tube arm;
an actuator configured to drive rotation of the tube shaft; and
a gearset including a driving gear coupled to the actuator and having at least one first tooth and a driven gear coupled to the tube shaft and having at least one second tooth, wherein the at least one second tooth of the driven gear is formed from a polymer material, and wherein the driven gear is over-meshed with the driving gear such that the at least one second tooth is in direct contact with the at least one first tooth with the at least one second tooth at least partially deflected causing a loading force within the at least one second tooth.

2. The x-ray imaging system of claim 1, wherein the deflection of the at least one second tooth includes bending and compression of the at least one second tooth.

3. The x-ray imaging system of claim 1, wherein the loading force is between 0.1 kilopound per square inch (ksi) and 5 ksi.

4. The x-ray imaging system of claim 1, wherein the loading force is measured when the x-ray tube arm is at a 0° tube arm angle.

5. The x-ray imaging system of claim 1, wherein the loading force occurs when the x-ray tube arm is between at least a ±5° and a ±30° tube arm angles.

6. The x-ray imaging system of claim 1, wherein the driving gear is formed from a metal material, the driving gear is a wormshaft and the at least one first tooth is a helical tooth.

7. The x-ray imaging system of claim 1, wherein the driving gear includes a metal core, the polymer material cast on the metal core and forming a tooth ring.

8. The x-ray imaging system of claim 1, wherein the rotational drive assembly includes a second tube shaft supporting the arm, a second actuator configured to drive rotation of the second tube shaft, and a second gearset, the second gearset having a smaller diameter driven gear than the driven gear of the tube shaft of the x-ray tube arm.

9. The x-ray imaging system of claim 1, wherein the driven gear is rotatable around the rotational axis and the driving gear is rotatable around a drive axis, and wherein a distance between the drive axis and the rotational axis is such that the driving gear has an interference fit with the driven gear and a first pitch radius of the at least one first tooth at least partially overlaps with a second pitch radius of the at least one second tooth.

10. The x-ray imaging system of claim 9, wherein the overlap of the first pitch radius of the at least one first tooth and the second pitch radius of the at least one second tooth compresses the at least one second tooth inducing the loading force therein.

11. The x-ray imaging system of claim 9, wherein the overlap of the first pitch radius of the at least one first tooth and the second pitch radius of the at least one second tooth is between 1% and 5% of the distance between the drive axis and the rotational axis at a 0° tube arm angle in a fully-meshed configuration.

12. The x-ray imaging system of claim 9, wherein the interference fit is measured when the x-ray tube arm is at a 0° tube arm angle.

13. The x-ray imaging system of claim 9, wherein the interference fit occurs when the x-ray tube arm is between at least a ±5° and a ±30° tube arm angles.

14. The x-ray imaging system of claim 9, wherein the driving gear is formed from a metal material, the driving gear is a wormshaft and the at least one first tooth is a helical tooth.

15. The x-ray imaging system of claim 9, wherein the driving gear includes a metal core, the polymer material cast on the metal core and forming a tooth ring.

16. The x-ray imaging system of claim 9, wherein the rotational drive assembly includes a second tube shaft supporting the arm, a second actuator configured to drive rotation of the second tube shaft, and a second gearset, the second gearset having a smaller diameter driven gear than the driven gear of the tube shaft of the x-ray tube arm.

17. A method of rotating an x-ray tube arm of an imaging system, the imaging system including a gantry rotationally supporting the x-ray tube arm and an arm having an immobilization system including a paddle, a support platform, and an x-ray receptor disposed below the support platform, the method comprising:
providing a rotational drive assembly, wherein the rotational drive assembly includes a tube shaft rotatable around a tube arm axis and supporting the x-ray tube arm, an actuator, and a gearset including a driving gear coupled to the actuator and having at least one first tooth and a driven gear coupled to the tube shaft and having at least one second tooth, wherein the at least one second tooth of the driven gear is formed from a polymer material, and wherein the driven gear is over-meshed with the driving gear such that a loading force is induced within the at least one second tooth;
positioning, via the rotational drive assembly, the x-ray tube arm that includes an x-ray source at a 0° tube arm angle relative to the gantry;
rotating the x-ray tube arm about the tube arm axis and out of the 0° tube arm angle by actuating the driving gear thereby rotating the driven gear around the tube arm axis; and
based on the loading force within the at least one second tooth, preventing lash of the x-ray tube arm via the driven gear when a weight of the x-ray tube arm moves out of the 0° tube arm angle.

18. The method of claim 17, further comprising inducing the loading force based on a force-displacement approach, a pressure approach, or a sensing approach.

19. The method of claim 17, wherein rotating the x-ray tube arm occurs during a tomosynthesis imaging mode of the imaging system.

20. The method of claim 17, wherein the loading force prevents lash of the x-ray tube arm at least between a ±5° and a ±30° tube arm angles.
